(19) United States
(12) Patent Application Publication
(10) Pub. No.: US 2007/0299360 A1
(43) Pub. Date: Dec. 27, 2007

(54) Systems and Methods for Analyzing and Assessing Dementia and Dementia-Type Disorders

(75) Inventors: Steven M. Snyder, Boulder, CO (US); James D. Falk, Duluth, MN (US)

Correspondence Address: SUTHERLAND ASBILL & BRENNAN LLP, 999 PEACHTREE STREET, N.E., ATLANTA, GA, 30309, US

(73) Assignee: LEXICOR MEDICAL TECHNOLOGY, LLC, Augusta, GA (US)

(21) Appl. No.: 11/766,615

(22) Filed: Jun. 21, 2007

Related U.S. Application Data
(60) Provisional application No. 60/815,373, filed on Jun. 21, 2006.

(51) Int. Cl. A61B 5/0476

(52) U.S. Cl. 600/544

(57) ABSTRACT

Embodiments of the invention can provide systems and methods for analyzing and assessing dementia and dementia-type disorders by integrating the use of electroencephalography (EEG), neuropsychological or cognitive testing data, and cardiovascular risk factor data. Embodiments of the invention can provide systems and methods for early detection of dementia, including Alzheimer's disease (AD), vascular dementia (VAD), mixed dementia (AD and VAD), MCI, and other dementia-type disorders. Embodiments of the invention can provide some or all of the following improvements over conventional systems and methods, including: (1) Increased sensitivity, specificity, and overall accuracy; (2) Detection of AD, VAD and mixed dementia; and (3) Accurate detection of mild dementia and some cases of mild cognitive impairment in addition to the detection of moderate to severe dementia.

### RELATED APPLICATION

This application claims priority to U.S. Provisional Ser. No. 60/815,373 entitled “Systems and Methods for Analyzing and Assessing Dementia.” which was filed on Jun. 21, 2006, the contents of which are hereby incorporated by reference.

### TECHNICAL FIELD

The invention relates to detection of biological disorders. More particularly, the invention relates to systems and methods for analyzing and assessing dementia and dementia-type disorders.

### BACKGROUND

The United States Congress Office of Technology Assessment estimates that as many as 6.8 million Americans suffer from mild to severe dementia. According to the Alzheimer's Association, about 4.5 million (or approximately two-thirds) of dementia patients are afflicted specifically with Alzheimer's disease (AD). Vascular dementia (VAD) is the second most common form of dementia, accounting for approximately one tenth to one-third of cases. Therefore, the estimated potential diagnosis and treatment market may be as much as US $1.4 billion if each potential patient is scanned or otherwise examined an average of one time.

By some estimates, the estimated market may be even larger. While some diagnostic assessments may require a single scan, tracking the treatment of a patient can require multiple scans.

One major risk factor for dementia is aging. Regular diagnostic assessments such as annual screenings could be prescribed to all adults at or above the age of 50, which would address a market of approximately 77 million Americans (based on year 2000 census estimates). As the life expectancy in America continues to increase and the baby boom generation ages, the number of people suffering from dementia is projected to increase correspondingly. For instance, one estimate provides that by the year 2040, the number of persons with Alzheimer's disease alone may exceed about 6 million.

There have been several published studies regarding linear and non-linear electroencephalography (EEG) diagnostic methods for detecting AD and the relative accuracy of such methods in diagnosing AD. Examples of these studies are Jeong (2002) and Jeong (2004). For example, using conventional linear-type diagnostic methods, overall diagnostic accuracy has been consistently observed at approximately 80% for AD versus normal controls (Jeong, 2004) with lower reported accuracy of about 65% for the detection of VAD (Renna et al., 2003). Typically, the diagnostic accuracy is relatively higher for severe AD cases, and the accuracy decreases with moderate and mild AD cases. Using non-linear-type complexity measures, prior studies have examined the EEC from patients with AD and report detection accuracies of about 70% (Jeong, 2002). At least one study has investigated the use of non-linear-type features of EEG with diagnosis of VAD (Jeong, 2001).

At least two United States patents, Nos. 5,230,346 and 5,309,923, relate to assessing AD and multi-infarct dementia (a common form of VAD) using linear-type EEG methods rather than non-linear-type techniques. The linear-type methods described by these patents include spectral ratio and coherence measures. Each of these patents relates to use of a primary measure, cordance, to target and locate brain lesions that are indicative of various pathologies. U.S. Pat. No. 5,230,346 discloses diagnostic accuracy rates of an estimated 79% sensitivity and 74% specificity by cross-validation.

Thus, there is a need for systems and methods for analyzing and assessing dementia and dementia-type disorders.

There is a further need for systems and methods for analyzing and assessing dementia and dementia-type disorders using non-linear type data and analysis.

### SUMMARY OF THE INVENTION

Embodiments of the invention can provide systems and methods for analyzing and assessing dementia and dementia-type disorders by integrating the use of electroencephalography (EEC), neuropsychological testing, and cardiovascular risk factors. Embodiments of the invention can provide systems and methods for relatively early detection of dementia and dementia-type disorders, including Alzheimer's disease (AD), vascular dementia (WAD), mixed dementia (AD and VAD), and mild cognitive impairment (MCI). Embodiments of the invention can provide some or all of the following improvements over conventional systems and methods, including: (1) Increased sensitivity, specificity, and overall accuracy; (2) Detection of AD, VAD and mixed dementia and other dementia-type disorders; and (3) Accurate detection of mild dementia and some cases of mild cognitive impairment in addition to the detection of moderate to severe dementia and other dementia-type disorders. Embodiments of the invention can utilize a non-linear-type analysis of EEG data rather than linear analysis, and statistically combine the results of non-linear-type EEG data analysis with measures of neuropsychological or cognitive testing and cardiovascular risk factors. Such embodiments can provide more reliable predictive information than use of linear-type EEG measures in conventional systems and methods.

In one embodiment, embodiments of the invention can utilize various statistical methods, for example, logistic regression, to integrate non-linear-type EEG results with results of neuropsychological testing, such as ADAS-Cog, the cognitive portion of the Alzheimer's Disease Assessment Scale, and cardiovascular risk factors based at least on medical history and/or MRI/CT (magnetic resonance imaging/computed tomography) results. The use of integrated and comprehensive test results in a diagnostic tool in accordance with an embodiment of the invention is capable of providing a probability that a particular subject is experiencing early to advanced stages of dementia.

In one aspect of an embodiment of the invention, results or output can be cross-validated with a clinical database. In one example, improvements in sensitivity can be increased to approximately 87% o and specificity can be increased to about 93% for AD and VAD, in comparison to the 79% sensitivity and 74% specificity obtained from conventional techniques.

In another aspect of an embodiment of the invention, a complexity measuring algorithm capable of determining a non-linear-type measure of EEC data can be implemented. This type of algorithm can utilize fewer consecutive EEG data points (fewer artifact-free epochs) than other algorithms. Such an embodiment may collect data from as few as a single electrode site, thus allowing for relatively faster electrode application and use of relatively inexpensive EEG equipment, thereby reducing cost and increasing efficiency.

In one embodiment, a method for analyzing a dementia-type disorder in a person can be provided. The method can include receiving a plurality of electroencephalography data associated with a person. In addition, the method can include receiving a plurality of cardiovascular risk factor data associated with the person. Further, the method can include receiving a plurality of cognitive data associated with the person. Moreover, the method can include determining an indication of whether the person is at risk for the dementia-type disorder based at least in part on a portion of the electroencephalography data, cardiovascular risk factor data, and cognitive data.

In one aspect of the embodiment, the plurality of electroencephalography data can include at least one of the following: electroencephalography data taken at a T5 electrode site for the person, electroencephalography data collected with the person's eyes open, electroencephalography data collected with the person's eyes closed, or a combination of electroencephalography data collected with the person's eyes open and closed.

In another aspect of the embodiment, at least a portion of the electroencephalography data is processed using at least one of the following: a fractal dimension methodology, or a box counting algorithm.

In another aspect of the embodiment, the plurality of cardiovascular risk factor data can comprise any factor indicative of a higher probability for the person eventually suffering from cardiovascular disease associated with a history of at least one of the following: stroke, transient ischemic attack, myocardial infarct, alcohol abuse, arterial bypass surgery, arterial blockage, hypertension, high cholesterol, diabetes, untreated diabetes, chronic obstructive pulmonary disease, emphysema, alcohol abstention, overweight, male gender, and not married (widowed, divorced, or single).

In another aspect of the embodiment, the plurality of cognitive data can include at least one of the following: an ADAS-Cog test score associated with the person, data associated with an ADAS-Cog test administered to the person, data associated with memory of the person, data associated with praxis of the person, or data associated with a language skill of the person.

In another aspect of the embodiment, the dementia-type disorder can include at least one of the following: Alzheimer's disease (AD), vascular dementia (VAD), mixed dementia (AD and VAD), or mild cognitive impairment (MCI).

In another aspect of the embodiment, the method can include receiving a plurality of other health data associated with the person, and based at least in part on a portion of the electroencephalography data, cardiovascular risk factor data, cognitive data, and other health data, determining an indication of whether the person is at risk for the dementia-type disorder, wherein the other health data can include at least one of the following: medical history of the person, health data collected from a questionnaire, brain imaging data, or genetic testing data.

In another embodiment of the invention, a system for analyzing a dementia-type disorder in a person can be provided. The system can include a data collection module adapted to receive a plurality of electroencephalography data associated with a person. The data collection module can be further adapted to receive a plurality of cardiovascular risk factor data associated with the person. In addition, the data collection module can be further adapted to receive a plurality of cognitive data associated with the person. The system can also include a report generation module adapted to determine an indication of whether the person is at risk for the dementia-type disorder based at least in part on a portion of the electroencephalography data, cardiovascular risk factor data, and cognitive data.

In one aspect of the embodiment, the data collection module is further adapted to receive a plurality of other health data associated with the person; and the report generation module is further adapted to determine an indication of whether the person is at risk for the dementia-type disorder based at least in part on a portion of the electroencephalography data, cardiovascular risk factor data, cognitive data, and other health data.

In another aspect of the embodiment, the data collection module is further adapted to output the indication comprising a probability against a receiver operating characteristic (ROC) curve comprising data associated with a clinical database.

In another aspect of the embodiment, the data collection module is further adapted to normalize some or all of the electroencephalography data.

In another aspect of the embodiment, the data collection module is further adapted to implement an averaging methodology to some or all of the electroencephalography data.

In another aspect of the embodiment, the data collection module is further adapted to implement a fractal dimension methodology to some or all of the electroencephalography data.

In another aspect of the embodiment, the data collection module is further adapted to implement a box counting algorithm to some or all of the electroencephalography data.

In another aspect of the embodiment, the data collection module is further adapted to implement a logistic regression model with some or all of the cognitive data.

In another aspect of the embodiment, the data collection module is further adapted to standardize some or all of the cognitive data using a normative database.

In another aspect of the embodiment, the data collection module is further adapted to implement a logistic regression model with some or all of the cardiovascular risk factor data.

In yet another embodiment of the invention, another system for analyzing a dementia-type disorder in a person can be provided. The system can include at least one data collector adapted to receive a plurality of electroencephalography data associated with a person. In addition, the data collector can be adapted to receive a plurality of cardiovascular risk factor data associated with the person. Furthermore, the data collector can be adapted to receive a plurality of cognitive data associated with the person. The system can also include at least one processor adapted to determine an indication of whether the person is at risk for the dementia-type disorder based at least in part on a portion of the electroencephalography data, cardiovascular risk factor data, and cognitive data. Further, the system can include at least one output device adapted to output the indication of whether the person is at risk for the dementia-type disorder.

In one aspect of the embodiment, the plurality of electroencephalography data can include at least one of the following: electroencephalography data taken at a T5 electrode site for the person, electroencephalography data collected with the person's eyes open, electroencephalography data collected with the person's eyes closed, or a combination of electroencephalography data collected with the person's eyes open and closed; wherein the plurality of cardiovascular risk factor data can include any factor indicative of a higher probability for the person eventually suffering from cardiovascular disease associated with a history of at least one of the following: stroke, transient ischemic attack, myocardial infarct, alcohol abuse, arterial bypass surgery, arterial blockage, hypertension, high cholesterol, diabetes, untreated diabetes, chronic obstructive pulmonary disease, emphysema, alcohol abstention, overweight, male gender, and not married (widowed, divorced, or single); and wherein the plurality of cognitive data can include at least one of the following: an ADAS-Cog test score associated with the person, data associated with an ADAS-Cog test administered to the person, data associated with memory of the person, data associated with praxis of the person, or data associated with a language skill of the person.

Other systems and processes according to various embodiments of the invention will become apparent with respect to the remainder of this document.

### BRIEF DESCRIPTION OF THE DRAWINGS

Embodiments of the invention can be better understood with reference to the following drawings.

FIG. 1 illustrates a system in accordance with an embodiment of the invention.

FIG. 2 illustrates histograms of example comparative diagnostic results for dementia and normal subjects obtained using an embodiment of the invention.

FIG. 3 illustrates a chart of example diagnostic results obtained using an embodiment of the invention.

FIG. 4 is a flowchart illustrating a process in accordance with an embodiment of the invention.

### DETAILED DESCRIPTION OF EMBODIMENTS OF THE INVENTION

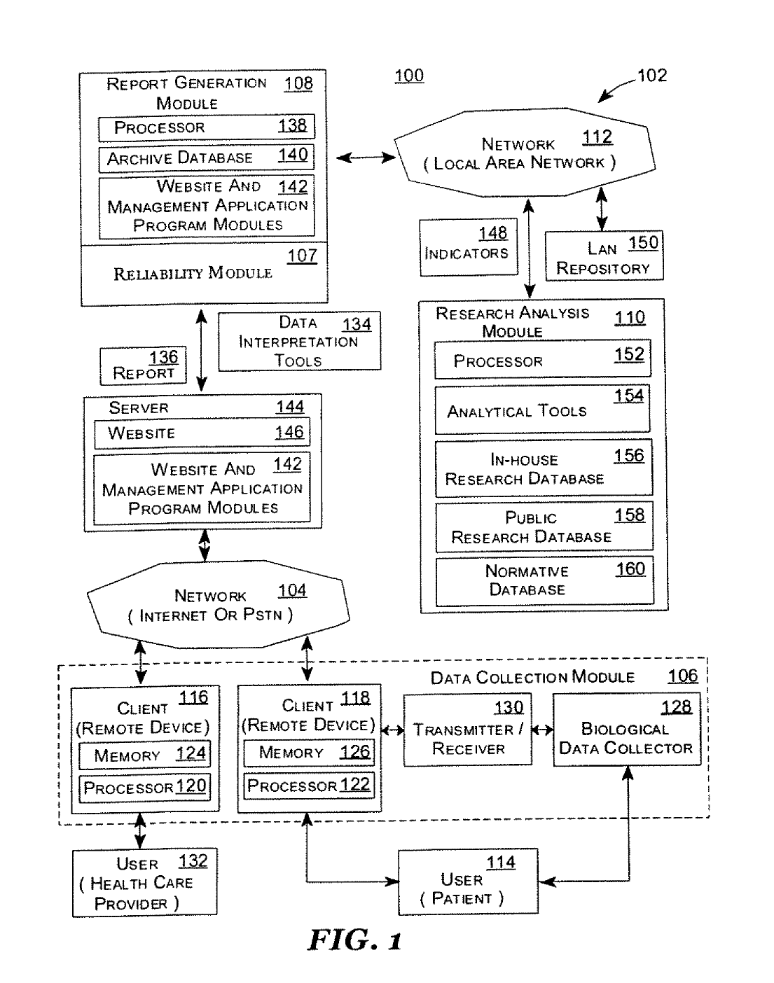

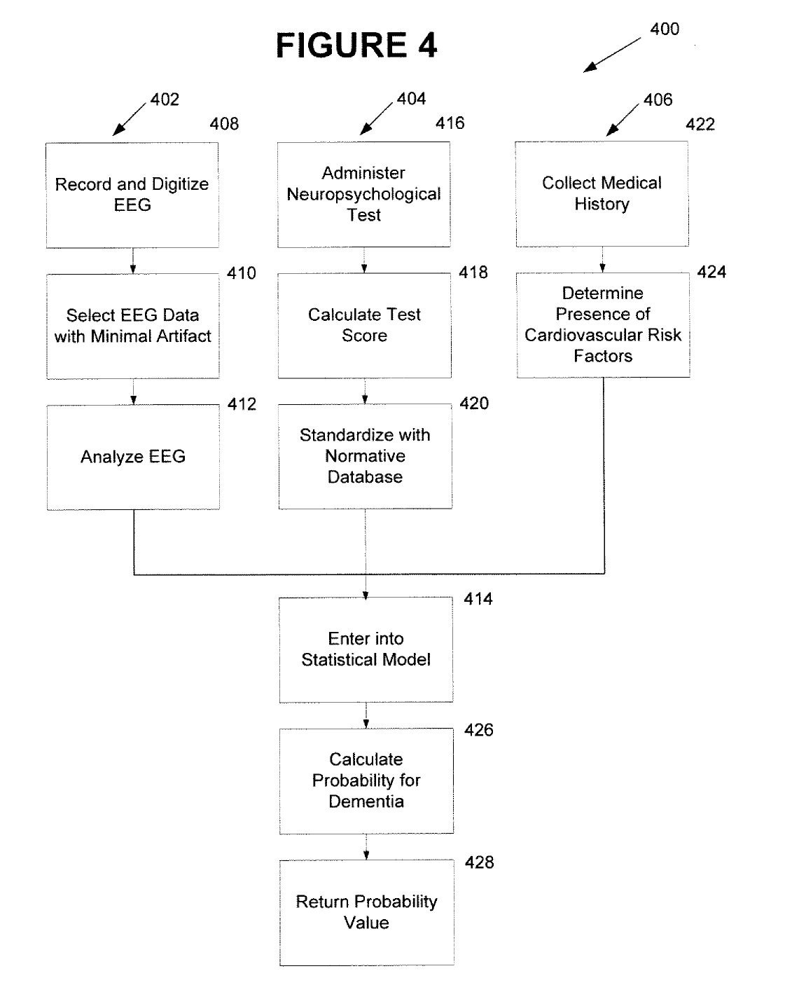

Systems for Analyzing and Assessing Dementia. FIG. 1 illustrates one example environment 100 for an example system 102 in accordance with an embodiment of the invention. Using the example system 102 illustrated in FIG. 1, the process of FIG. 4 can be implemented.

The environment 100 shown includes a network 104 in communication with the system 102. In turn, the system 102 includes one or more system modules, such as 106, 107, 108, 110, that can operate with and in accordance with various embodiments of the invention. Each of the system modules, for instance 106, 107, 108, 110, can communicate with each other through the network 104 or via an associated network 112 such as a local area network (LAN). For example in the embodiment shown, the system modules can be a data collection module 106, a frequency spectrum/reliability module 107, a report generation module 108, and a research analysis module 110. The data collection module 106 and frequency spectrum/reliability module 107 can communicate with the report generation module 108 via the Internet or a network such as 104, and the research analysis module 110 can communicate with the report generation module 108 via a LAN, such as 112. Other system modules in various configurations operating in accordance with embodiments of the invention may exist. The configuration and arrangement of the system modules 106, 107, 108, 110 are shown by way of example only, and other configurations and arrangements of system modules can exist in accordance with other embodiments of the invention.

Each of the system modules, such as 106, 107, 108, 110, can be hosted by one or more processor-based platforms such as those implemented by Windows 98, Windows NT/2000, LINUX-based and/or UNIX-based operating platforms. Furthermore, each of the system modules, such as 106, 107, 108, 110, can utilize one or more conventional programming languages such as DB/C, C, C++, UNIX Shell, and Structured Query Language (SQL) to accomplish various methods, routines, subroutines, and computer-executable instructions in accordance with the invention, including system functionality, data processing, and communications between functional components. Each of the system modules 106, 107, 108, shown in this embodiment, and their respective functions are described in turn below.

The data collection module 106 is adapted to collect biological data from a user such as a patient 114, person, or individual. In some instances, the data collection module 106 can receive or otherwise collect biological data from a user such as a health care provider 132, who may input data associated with a user such as a patient 114, person, or individual. In one example, biological data can include electroencephalography, electroencephalographic, qEEG, or EEG data (collectively known as “EEG data”) from a patient, such as 114. The data collection module 106 includes one or more clients 116, 118 and/or remote devices in communication with the network 104 such as the Internet. Typically, each client 116, 118 is a processor-based platform such as a personal computer, personal digital assistant (PDA), tablet, or other stationary or mobile computing-type device adapted to communicate with the network 104. Each client 116, 118 can include a respective processor 120, 122, memory 124, 126 or data storage device, biological data collector 128, and transmitter/receiver 130. Other components can be utilized with the data collection module 106 in accordance with other embodiments of the invention.

The biological data collector 128 can communicate with at least one client 116, 118 via a transmitter/receiver 130. In the embodiment shown, a biological data collector 128 such as a medical device can obtain or otherwise receive biological data in real-time, or near real-time, from a user such as a patient 114. The transmitter/receiver 130 can transmit the received biological data from the biological data collector 128 or medical device to the client 118. In turn, the client 118 may temporarily store the biological data in memory 126 or otherwise process the data with the processor 122, and further transmit the data via the network 104 to the reliability module 107 and/or report generation module 108. In other embodiments, a biological data collector 128 may locally store and process collected data, and communicate the data directly to the reliability module 107 and/or report generation module 108 via the network 104.

For example, a biological data collector 128 can be a medical device such as a Lexicor Digital Cortical Scan quantitative electroencephalographic (QEEG) data acquisition unit and Electrocap (collectively referred to as “DCS device”) to be provided by Lexicor Medical Technology, LLC. This type of medical device and associated configuration can be connected to a user or patient's head, and when activated, the medical device can provide digitized EEG data via a proprietary digital interface and associated software that permits data to be stored locally in a file format such as a Lexicor file format on a host platform. In alternative embodiments, data can be transmitted in real-time via other interfaces such as USB to the host platform such as a server. Stored EEG data can be uploaded to an associated server or client as needed. In other instances, collected or stored data can be burned onto or otherwise stored in a digital format such as a CD-R disk and then transmitted or transferred to an associated server or client.

Note that a Lexicor file format can be a Lexicor raw EEG data file format to be developed by Lexicor Medical Technology, LLC. This particular file format has a data structure that is adapted to store 24 channels of digitized EEG data to facilitate offline data analysis. Although various EEG storage formats exist, the Lexicor file format can be adapted to handle these and other data storage formats. For example, the Lexicor file format has a global header with 64 integers to handle information such as sample rate, gain of the front end DCS amplifiers, software revision, an total number of epochs. Further, the Lexicor file format can include one or more epochs or sections of raw data including a 256 byte text array to handle comment entries, as well as an array to handle raw digitized EEG data collected by a DCS device during a particular acquisition period for a particular epoch, and a local header containing the epoch number and status of the particular epoch.

A biological data collector 128 can also include, but is not limited to, blood pressure monitors, weight scales, glucose meters, oximeters, spirometers, coagulation meters, urinalysis devices, hemoglobin devices, thermometers, capnometers, electrocardiograms (EKGs), electroencephalagrams (EEGs), other digital medical devices that can output data via a RS-232 port or similar type connection, and other devices or methods that can provide data associated with a biological, neurophysiological or cognitive, or other physiological function. Biological data collected or otherwise received from a user, patient, or individual can include, but is not limited to, blood pressure, weight, blood component measurements, bodily fluid component measurements, temperature, heart measurements, brainwave measurements, and other measurements associated with a biological, neurophysiological or cognitive, or physiological function.

The transmitter/receiver 130 typically facilitates the transfer of data between the biological data collector 128 and client 118. The transmitter/receiver 130 can be a stand alone or built-in device. The transmitter/receiver 130 can include, but is not limited to, a RS-232 compatible device, a wireless communication device, a wired communications device, or any other device or method adapted to communicate biological data.

A user such as a healthcare provider 132 can share or separately utilize a client 116, 118 to interact or communicate with the network 104 depending upon the proximity of the client 116, 118 to the patient 114. The healthcare provider 132 and/or patient 114 may receive specific instructions from the report generation module 108 via the same or a respective client 116, 118. For example, in response to a particular condition, the report generation module 108 may request that from the health care provider 132 that specific biological data be collected from the patient 114. Appropriate instructions may be communicated to the health care provider 132 via the network 104 to the client 116. The health care provider 132 can then instruct the patient 114 or otherwise assist the patient 114 in connecting the biological data collector 128 or medical device to the patient 114. When activated, the biological data collector 128 or medical device can transmit biological data associated with the patient 114 via the network 104 or Internet to the report generation module 108. As needed, a healthcare provider 132, and/or patient 114, or other user can input demographic data or otherwise provide demographic data via a respective client 116, 118.

In one embodiment, a data collection module, such as 106, can be adapted to collect EEG data from a user or patient 114. Such data can be collected or otherwise received via a biological data collector, such as 128, or other type of data collector in communication with a user or patient, such as 114. Suitable EEG data can include, but is not limited to, electroencephalography data taken at a T5 electrode site for the patient, electroencephalography data collected with the patient's eves open, electroencephalography data collected with the patient's eyes closed, and a combination of electroencephalography data collected with the patient's eyes open and closed.

In one embodiment, a data collection module, such as 106, can be adapted to collect cognitive or neuropsychological data from a user or patient 114. Such data can be collected or otherwise received via a client or remote device, such as 116 or 118, or other type of data collector. A user such as a health care provider 132 or patient 114 can enter data via a corresponding client or remote device, such as 116 or 118, and the data can be stored and processed for subsequent use. Suitable cognitive or neuropsychological data can include, but is not limited to, an ADAS-Cog test score associated with the person, data associated with an ADAS-Cog test administered to the person, data associated with memory of the person, data associated with praxis of the person, or data associated with a language skill of the person.

In one embodiment, a data collection module, such as 106, can be adapted to collect medical history data comprising cardiovascular risk factor data from a user or patient 114. Such data can be collected or otherwise received via a client or remote device, such as 116 or 118, or other type of data collector. A user such as a health care provider 132 or patient 114 can enter data via a corresponding client or remote device, such as 116 or 118, and the data can be stored and processed for subsequent use. Suitable cardiovascular risk factor data can include, but is not limited to, any factor indicative of a higher probability for the person eventually suffering from cardiovascular disease associated with a history of at least one of the following: stroke, transient ischemic attack, myocardial infarct, alcohol abuse, arterial bypass surgery, arterial blockage, hypertension, high cholesterol, diabetes, untreated diabetes, chronic obstructive pulmonary disease, emphysema, alcohol abstention, overweight, male gender, and not married (widowed, divorced, or single).

In one embodiment, a data collection module, such as 106, can be adapted to collect other health data from a user or patient 114. Such data can be collected or otherwise received via a biological data collector, such as 128, client or remote device, such as 116 or 118, or other type of data collector in communication with a user or patient, such as 114. Suitable health data can include, but is not limited to, medical history of the person, health data collected from a questionnaire, brain imaging data, or genetic testing data. For instance, a data collection module such as 106 can implement a questionnaire for a user, healthcare provider 132, or patient 114 to complete. The questionnaire may be displayed via a client or remote device, such as 116, 118, and the user, healthcare provider 132, or patient 114 may input other health data in response to one or more prompts or questions provided by the questionnaire.

The frequency spectrum/reliability module 107 can be adapted to receive biological data from the data collection module 106, and to process some or all of the biological data to determine one or more reliability indexes based in part on at least some or all of the biological data. In the embodiment shown, a frequency spectrum/reliability module 107 can be a set of computer-executable instructions such as a software program stored on a server such as 144, or another processor-based platform such as a client device in communication with a server. The frequency spectrum/reliability module 107 shown can be integrated with the report generation module 108. In another embodiment a frequency spectrum/reliability module 107 can be a separate stand alone module with an associated processor such as an apparatus or reliability device. In another embodiment, a frequency spectrum/reliability module 107 can be an incorporated sub-system module for an associated website and management administration program module such as 142. As needed, various reports can be generated by a frequency spectrum/reliability module 107, and provided to a user, such as a health care provider 132.

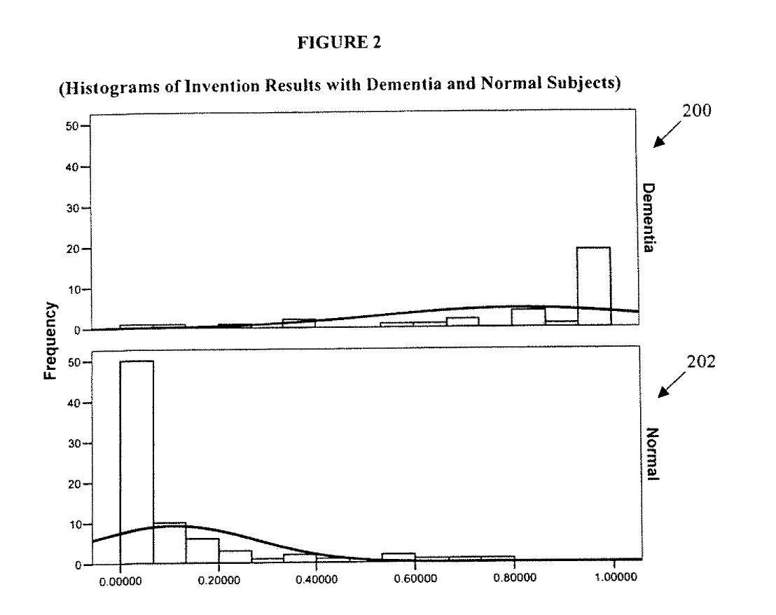

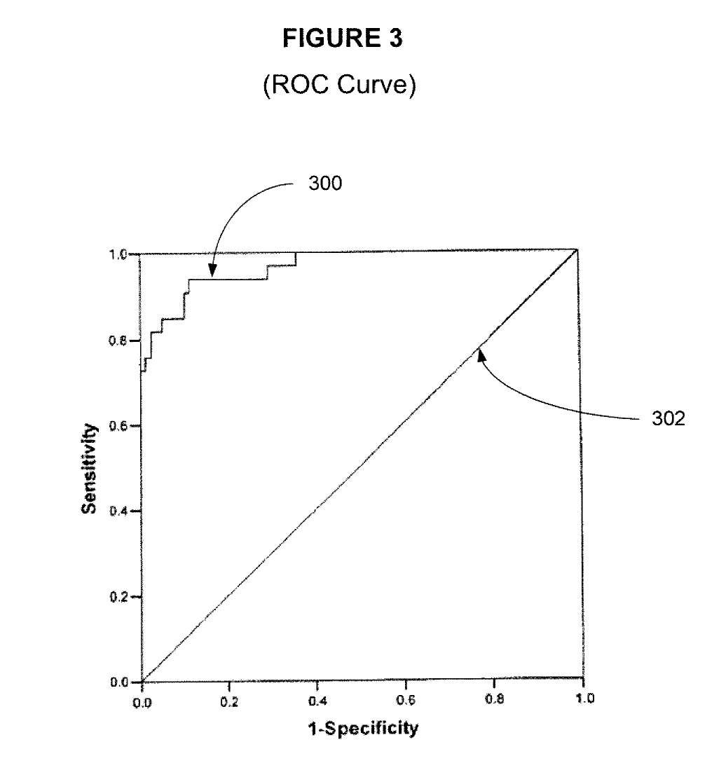

The report generation module 108 can be adapted to receive, store, and process the biological data from the patient 114 for subsequent retrieval and analysis. The report generation module 108 can also be adapted to generate one or more data interpretation tools 134 based upon collected or otherwise received biological data from the patient 114. Further, the report generation module 108 can be adapted to generate a report 136 including one or more data interpretation tools to assist a user such as a health care provider 132 in managing and analyzing biological data. An example data interpretation tool and report are described in greater detail with respect to FIGS. 2-3. In addition, the report generation module 108 can be adapted to operate in conjunction with or otherwise execute an associated website and management application program module 142.

Typically, the report generation module 108 can be a processor-based platform such as a server, mainframe computer, personal computer, or personal digital assistant (PDA). The report generation module 108 includes a processor 138, an archive database 140, and a website and management application program module 142. A separate server 144 to host an Internet website 146 can be connected between the report generation module 108 and the network 104 or Internet; or otherwise be in communication with the report generation module 108 and data collection module 106 via the network 104 or Internet. Generally, the separate server 144 can be a processor-based platform such as a server or computer that can execute a website and management application program module 142. In any instance, the report generation module 108 can communicate with the data collection module 106 via the network 104 or Internet. Other components can be utilized with the report generation module 108 in accordance with other embodiments of the invention.

In one embodiment, the report generation module 108 and other modules such as 106, 107, 110, 142, can include a set of computer-executable instructions or an associated computer program. The various sets of computer-executable instructions or computer programs can be processed by one or more associated processors, such as 138, or other computer hardware. Those skilled in the art will recognize the various embodiments for such modules and the implementation of these modules in accordance with the invention.

In one embodiment of the invention, the report generation module 108 can implement a set of computer-executable instructions or an associated computer program to process a combination of at least three different factors or types of data that, when input into a logistic regression model, can produce an output of a probability that a particular subject is suffering from the early stage of dementia (such as Alzheimer's disease (AD), vascular dementia (VAD), mixed dementia (AD and VAD), and Mild Cognitive Impairment (MCI)), or other dementia-type disorder. Various output from the system, such as 102, and/or the report generation module 108 can also be used to detect the advanced or later stages of dementia or other dementia-type disorders. In one example, a report generation module, such as 108, can utilize at least three factors or types of data such as the dimensional complexity of the subject's EEG data (measured by the fractal dimension), the existence of one or more specific cardiovascular risk factors associated with dementia, and the cognitive section of the Alzheimer's Disease Assessment Scale (ADAS-Cog). In another embodiment, a report generation module, such as 108, can implement an additional factor or type of data such as brain imaging (MRI/CT) data indicating evidence for cardiovascular disease. In a further embodiment, a report generation module, such as 108, can implement an additional factor or type of data such as specific genetic results and/or family history of similar disorders. Other factors, evidence, or data can be implemented as additional factors in combination with some or all of the factors or types of data described above.

Embodiments of the invention can combine results of EEG data and various types of clinical data to improve the prediction of a primary diagnosis of AD and/or VAD with mild to severe severities as well as MCI. These embodiments can integrate various statistical data to provide a prediction of dementia or dementia-type disorder diagnosis. Using logistic regression, a report generation module such as 108, can integrate data such as non-linear analysis of EEC data, neuropsychological test results covering memory, language, and praxis, and cardiovascular risk factors to provide a prediction of dementia or dementia-type disorder diagnosis. For example, using a logistic regression model, stepwise selection can be used with a wide array of risk factors, risk factor data, neuropsychological and cognitive data, and other clinical data as well as MRI and linear and non-linear analyses of EEG data to determine an optimal model. Other factors, types of data, or variables can be used in a logistic regression model or other models in accordance with other embodiments of the invention.

In one embodiment, a report generation module such as 108 can be adapted to receive EEG data and select certain EEG data with minimal artifacts for further analysis. In this embodiment, a report generation module, such as 108, can implement a set of computer-executable instructions or an associated computer program to screen collected EEG data for any artifacts, and if needed, modify or remove any affected epochs. Various devices, techniques, and methodologies can be used by the report generation module such as 108 to screen collected EEG data for any artifacts, and if needed, modify or remove any affected epochs.

In one embodiment, a report generation module such as 108 can be adapted to implement at least one averaging-type methodology with collected EEG data. In this embodiment, a report generation module such as 108 can implement a set of computer-executable instructions or an associated computer program to implement a fractal dimension method to process the collected EEG data. One suitable algorithm for use with a fractal dimension method is a box counting (BC) algorithm.

The processor 138 can handle biological data and/or demographic data received from the data collection module 106, or received via the frequency spectrum/reliability module 107. The processor 138 and/or the frequency spectrum/reliability module 107 can store the biological data and demographic data in the archive database 140 for subsequent retrieval, and/or process the biological data using other data received from the research analysis module 110. Typically, the processor 138 and/or the frequency spectrum/reliability module 107 can analyze biological data and/or demographic data from the data collection module 106 and can remove unwanted artifacts from the data. Relevant biological data and/or demographic data can be stored in the archive database 140 or other data storage device until needed. Using one or more indicators 148 received from the research analysis module 110 or otherwise generated or stored by the system 102, the processor 138 can process the biological data and/or demographic data to generate one or more data interpretation tools 134. The processor 138 can generate a report 136 including one or more indicators 148 and associated data interpretation tools 134 for transmission via the network 104 to a user such as the health care provider 132 and/or patient 114.

Data interpretation tools 134 can add relevant information and context to biological and/or demographic data in a report 136, such that the data can be more readily interpreted by a user such as a health care provider 132 to determine the state of a particular condition with a particular patient 114. Data interpretation tools 134 typically include patterns of biological and/or demographic data for normal subjects and subjects with the condition. The patterns of biological and/or demographic data can be presented in a report 136 which can include graphs and text. These patterns are determined from a meta-analysis of the body of scientific literature, and analysis of relevant databases for normal subjects as well as those with a particular condition and those with related conditions.

In one embodiment, biological data such as electroencephalography data or EEG data can be received or collected by the data collection module 106. The data collection module 106 can transmit the data to the report generation module 108, and the report generation module can process the data. Using processed data, various histograms, receiver operating characteristic curves (ROC), characteristics, aspects, qualities, indicators, or other indications can be generated to compare and analyze different populations and samples of patients. In the embodiment shown, a report generation module such as 108 can further generate an output such as the histogram and ROC curve shown and described as 200, 300 respectively in FIGS. 2 and 3.

The archive database 140 can be a database, memory, or similar type of data storage device. The archive database 140 is adapted to store biological data such as medical images, medical data and measurements, and similar types of information, as well as demographic data as previously described. Generally, the archive database 140 can be utilized by the report generation module 108 to store biological data and demographic data until called upon.

The website and management application program module 142 can typically be a set of computer-executable instructions adapted to provide a website 146 with at least one functional module to handle data communication between the website 146 and at least one user such as a health care provider 132 and/or patient 114. The website and management application program module 142 can be hosted by the report generation module 108, separate server, and/or a storage device in communication with the network 104. A website and management application program module 142 can include, but is not limited to, a main login module, a patient management module, a patient qualification module a patient assessment module, a patient care plan module, a data analysis module, a filter module, an import/export module, a virtual private network electronic data interchange (VPI EDI) module, a reporting module, an indicator report notification module, an indicator report delivery module, an administrative module, a notification (data filter/smart agent) administration module, a database module, and other similar component or functional modules. Other component modules associated with the website and management application program module 142 can operate in accordance with other embodiments of the invention.

The separate server 144 can be adapted to host the website 146 viewable via the Internet with a browser application program. Alternatively, the separate server 144 may host a website and management application program module 142 as well. A website 146 can provide communication access for a health care provider 132 and/or patient 114 to the report generation module 108. For example, a report 136 generated by the report generation module 108 may be posted to the website 146 for selective access and viewing via the network 104 or Internet by a user such as a health care provider 132 and/or patient 114 operating the same or a respective client 116, 118 via the network 104. In other instances, a report 136 may be transmitted by the report generation module 108 to a user such as a health care provider 132 and/or patient 114 via an electronic mail message communication, a telecommunications device, messaging system or device, or similar type communication device or method. An example of a report with a ROC curve generated in accordance with various embodiments of the invention is illustrated and described in detail below in FIG. 3.

The associated network 112 can typically be a local area network (LAN) that provides communications between the report generation module 108 and the research analysis module 110. A LAN repository 150 may be connected or otherwise accessible to the associated network 112 for additional storage of biological data, indicators, or other data collected, generated, or otherwise received by the system 102.

The research analysis module 110 can be adapted to obtain and collect relevant research materials and data. Furthermore, the research analysis module 110 can be adapted to process relevant research materials and data, and can be further adapted to determine one or more indicators 148 for a particular condition. Moreover, in one embodiment, the research analysis module 110 can be adapted to provide indicators 148 to the report generation module 108 in response to a particular patient's condition or collected biological, clinical, and demographic data. Typically, the research analysis module 110 can be a processor-based platform such as a server, mainframe computer, personal computer, or personal digital assistant (PDA). The research analysis module 110 can include a processor 152, analytical tools 154, an in-house research database 156, a public research database 158, and a normative database 160. Other components can be utilized with the research analysis module 110 in accordance with the invention.

The processor 152 can handle research and data collected or otherwise received by the research analysis module 110. The processor 152 can index and/or store the research or data in an associated database for subsequent retrieval, or processes the research and data using one or more analytical tools 154. One or more indicators 148 can be provided or otherwise derived by or from the analytical tools 154, and the processor 152 can transmit any indicators 148 to the report generation module 108 as needed.

At least one analytical tool 154 can be utilized by the research analysis module 110, Typically, an analytical tool 154 can be an algorithm that utilizes research and data to determine one or more indicators 148 for a particular condition.

The in-house research database 156 can be a collection of research and articles provided by a particular or third-party vendor. Typically, an entity operating the system 102 can provide its own research and articles for a range of conditions. For example, information available from an in-house research database includes, but is not limited to, electronic databases, scientific and research journals, on-line sources, libraries, standard textbooks and reference books, and on-line and printed statements of committees and boards, and the like.

The public research database 158 can be a collection of research and articles provided by one or more third-parties. Typically, research and articles are available for free or upon payment of a fee from a variety of on-line or otherwise accessible sources. For example, information available from a public research database 156 includes, but is not limited to, electronic databases, scientific and research journals, on-line sources, libraries, standard textbooks and reference books, on-line and printed statements of committees and boards, and the like.

The normative database 160 can be a collection of electronic databases, scientific and research journals, on-line sources, libraries, standard textbooks and reference books, on-line and printed statements of committees and boards, and the like.

Another example system to collect and analyze EEG data measurements for analyzing and assessing dementia or dementia-type disorders in a user, patient, or an individual will be implemented by Lexicor Medical Technology, LLC. of Augusta, Ga. Other suitable systems and components to collect EEG data measurements have been disclosed in U.S. Ser. No. 11/565,305, filed Nov. 30, 2006, entitled “Systems and Methods for Analyzing and Assessing Depression and Other Mood Disorders Using Electroencephalography (EEG) Measurements”; U.S. Ser. No. 11/053,627, entitled “Associated Systems and Methods For Managing Biological Data and Providing Data Interpretation Tools,” filed Feb. 8, 2005, which is a continuation-in-part of U.S. Ser. No. 10/368,295, entitled “Systems and Methods For Managing Biological Data and Providing Data Interpretation Tools,” filed Feb. 18, 2003, which claims priority to U.S. Provisional Patent Application No. 60/358,477, filed Feb. 19, 2002. Other system embodiments in various configurations and including other components operating in accordance with other embodiments of the invention may exist.

In one embodiment, a data collection module, such as 106 in FIG. 1, can receive EEG data as described above in FIG. 1. The data collection module can operate in conjunction with a report generation module, such as 108 in FIG. 1, to process the EEG data in accordance with some or all of the methods, processes, procedures, and techniques described above. The report generation module 108 can include associated reporting and communication functionality to provide electronic and/or printed report formats to a variety of health care providers, professionals, researchers, or other users. In one embodiment, various report formats can be provided via a network, such as the Internet or network 104 in FIG. 1.

An example comparative summary of various conventional techniques with an embodiment of the invention is shown in Table 1 below. Each row of Table 1 represents an application of a particular logistic regression model. All of the models shown in Table 1 were applied to detect AD and/or VAD with mild to severe severities as well as MCI in a population aged 50 to 85 (N=111; 33 dementia patients and 78 age-matched adults). A relatively higher value of R² in the fourth column of Table 1 indicates that more of the variation of the medical diagnosis of dementia versus normal adults is explained by the model, from a minimum of 0 (0% of variation) to a maximum of 1 (100% of variation); and a relatively higher overall accuracy in the fifth column is an indicator of both relatively higher sensitivity and specificity. As shown, the relative overall accuracy of each conventional technique progressively increases from about 65% to about 80%, with the highest overall accuracy (approximately 92%) associated with an embodiment of the invention implementing and integrating a non-linear-type analysis of EEG data, the existence of one or more specific cardiovascular (CV) risk factors associated with dementia, and the cognitive section of the Alzheimer's Disease Assessment Scale (ADAS-Cog).

TABLE 1
Comparative Summary of Conventional Techniques with an
Embodiment of Invention

| Neuropsychological | Medical | | | Overall |
|---|---|---|---|---|
| Testing | History | Biological Data | R² | Accuracy |
| ADAS-Cog | none | none | 0.29 | 65% |
| ADAS-Cog | CV risk | none | 0.32 | 70% |
| | factors | | | |
| ADAS-Cog | CV risk | MRI/CT | 0.32 | 70% |
| | factors | | | |
| ADAS-Cog | CV risk | Linear analysis of | 0.66 | 85% |
| | factors | EEG | | |
| ADAS-Cog | CV risk | Non-linear analysis | 0.84 | 92% |
| | factors | of EEG | | |

Key to Abbreviations:
ADAS-Cog, cognitive portion of the Alzheimer's Disease Assessment Scale;
CV, cardiovascular;
CT, Computed Tomography;
MRI, Magnetic Resonance Imaging;
EEG, Electroencephalography.

It can be seen in Table 1 that the inclusion of cardiovascular risk factors together with neuropsychological testing can provide a general improvement in overall accuracy and R² value. The addition of MRI/CT data to cardiovascular risk factors and neuropsychological testing can provide relatively little or no improvement to overall accuracy and R², as shown in the third row, when compared to the model in the second row. The addition of linear-type analysis EEG data to cardiovascular risk factors and neuropsychological testing can provide an improvement in overall accuracy and R², as shown in the fourth row, when compared to the models in the first, second, and third rows. The non-linear-type analysis of EEG data when integrated with cardiovascular risk factors and neuropsychological testing can provide a relatively greater improvement to overall accuracy and R² when compared to all other models in Table 1.

In the above embodiment utilizing non-linear-type analysis of EEG data, the neuropsychological testing utilized was ADAS-Cog. In other embodiments, any suitable measure of memory, language, praxis or other measure of neuropsychological testing can be used. Furthermore, for the above embodiment, specific cardiovascular (CV) risk factors such as history of stroke, transient ischemic event, myocardial infarct, alcohol abuse, arterial bypass surgery and/or significant arterial blockage were selected by statistical analysis, in other embodiments, other suitable types of similar risk factors can be used as predictive values and can function in a similar capacity, such as hypertension, high cholesterol, diabetes, untreated diabetes, chronic obstructive pulmonary disease, emphysema, alcohol abstention, overweight, male gender, and not married (widowed, divorced, or single).

In the analyses illustrated in Table 1, MRI/CT was compared against different analyses of EEG data, and the non-linear analysis of EEG data (recorded at site T5 with eyes closed) provided greater relative predictive accuracy. Other embodiments of the invention can implement EEG from other electrode sites or any other combination of sites as well as with other types of suitable recording requirements and analytical techniques.

In another embodiment, neuropsychological testing and non-linear EEG data were implemented with MRI/CT instead of cardiovascular risk factors with an R² value of about 0.79 and an overall accuracy of approximately 88%. In some instances, the MRI/CT information and cardiovascular risk factor information may overlap, and one can be used in place of the other in other embodiments of the invention. In other instances, cardiovascular risk factors can be used rather than MRI/CT information due to the improved overall accuracy as well as clinical practicality, i.e., it can be more efficient and cost effective for a health care provider, professional or other person to determine cardiovascular risk factors by referencing a subject's previous medical history and/or evaluating a patient's questionnaire rather than collecting a new set of MRI or CT data to detect any abnormalities or characteristics indicative of cardiovascular disease.

Predicted Probability for Embodiments of the Invention. As can be observed in the histograms of FIG. 2, embodiments of the invention can separate a majority of a dementia sample (AD, VAD, mixed dementia, and MCI) from a majority of a normal population. The histograms shown in FIG. 2 utilize a sample of dementia patients and normal population of adults aged about 50-85 (N=11).

Referring to the histograms 200, 202 of FIG. 2, each evaluated individual can receive a calculated probability of 0 to 1 predicting membership with the dementia population. The evaluated dementia sample shown can span in severity from mild cognitive impairment to severe dementia, and can include AD and VAD as dementia subtypes. A user such as a qualified clinician can use a receiver operating characteristic (ROC) curve 300 shown in FIG. 3 and associated tabulated results to interpret the data. The data can provide sensitivity and specificity values for each selected probability cutoff. The ROC curve 300 shown in FIG. 3 has been derived from a clinical database.

In this example, the ROC curve 300 is shown above a diagonal reference line 302. Generally, the further above the ROC curve 300 is above the reference line 302, the greater the accuracy. Quantitatively, the area under the ROC curve 300 shown is about 0.967, which represents the probability that the result for a randomly chosen dementia patient will exceed the result for a randomly chosen normal adult.

A sample of the tabulated results by probability cutoff for the ROC curve 300 shown is presented in Table 2.

TABLE 2
ROC Results by Cutoff

| Probability of Dementia | | |
|---|---|---|
| Greater Than or Equal To: | Sensitivity | 1 − Specificity |
| .0000000 | 1.000 | 1.000 |
| .0063473 | 1.000 | .821 |
| .0145871 | 1.000 | .603 |
| .0149825 | 1.000 | .577 |
| .0192227 | 1.000 | .551 |
| .0293244 | 1.000 | .526 |
| .0354062 | 1.000 | .500 |
| .0360116 | 1.000 | .474 |
| .0381704 | 1.000 | .449 |
| .0447128 | 1.000 | .423 |
| .0.519001 | 1.000 | .397 |
| .0543357 | 1.000 | .372 |
| .0682979 | .970 | .359 |
| .0788769 | .970 | .333 |
| .1171722 | .970 | .308 |
| .2657227 | .939 | .115 |
| .3388205 | .909 | .103 |
| .5480675 | .848 | .051 |
| .6541104 | .818 | .026 |
| .6738702 | .758 | .013 |
| .7111013 | .727 | .013 |
| .7819441 | .727 | .000 |
| .8206846 | .697 | .000 |
| .9322908 | .576 | .000 |
| .9663375 | .515 | .000 |
| .9918616 | .455 | .000 |
| .9995869 | .273 | .000 |
| 1.0000000 | .000 | .000 |

With logistic regression modeling, the standard cutoff is at a calculated probability of about 0.5. As shown in the above embodiment, the sensitivity is about 87%, specificity about 93%, and overall accuracy approximately 91% as determined by random split half cross-validation with the clinical database (N=111). These values are an example representation of the performance of an embodiment of the invention using a logistic regression model performed with separate sample populations.

For further reference, the predictive accuracies for dementia diagnosis by embodiments of the invention for normal adults and subtypes of dementia are shown in Table 3 using individual probabilities derived for the total database.

TABLE 3
Predictive Accuracy by Group

| Group | Accuracy |
|---|---|
| Normal Adults | 94% |
| Alzheimer's Disease | 100% |
| Vascular Dementia | 73% |
| Mixed Dementia (AD and VAD) | 100% |
| Mild Cognitive Impairment | 60% |
| Total | 91% |

Methods for Analyzing and Assessing Dementia. Embodiments of the invention can provide systems and methods for analyzing and assessing dementia and dementia-type disorders, including the method 400 described below with respect to FIG. 4. In the embodiment of FIG. 4, at least three data collection sub-processes can be utilized, including an EEC data collection and analysis sub-process 402, a neuropsychological or cognitive data collection and analysis sub-process 404, and a medical history or risk factor data collection and analysis sub-process 406. Other embodiments of the invention can include some or all of these sub-processes, or other sub-processes. Furthermore, some or all of the elements described below with respect to the sub-processes can be utilized with other methods in accordance with other embodiments of the invention without regard to the order of the elements or the order of performing each of the sub-processes.

EEG Data Collection and Analysis. As shown in FIG. 4, the method 400 includes several sub-processes, including an EEG data collection and analysis sub-process 402, a neuropsychological or cognitive data collection and analysis sub-process 404, and a medical history or risk factor data collection and analysis sub-process 406.

The EEG data collection and analysis sub-process 402 begins at block 408. In block 408, EEG data from a subject is recorded and digitized by a system, such as 102 in FIG. 1. In this embodiment, an electrode associated with a system, such as 102, or biological data collector, such as 128, can be placed at site T5 of a subject's body, located for instance, using the International 10-20 system of electrode placement. In other embodiments, an electrode or other device can be placed on other portions of a subject's body. In other embodiments, EEG data can be collected via other suitable devices, techniques, or methodologies.

In addition, the area of the subjects body can be cleaned using a suitable EEG data preparation cleaner and alcohol. Once the electrode is properly or otherwise suitably placed, a syringe can be used to apply a conductive gel to the subject's body, for instance his or her scalp, in the selected site. The site on the subject's body can be verified to make sure that an accurate or otherwise suitable measurement can be obtained from that site.

EEG data collection can be performed during time periods with the subject's eyes closed and the subject's eyes opened. For example, EEG data can be collected for about 10 minutes with the subject's eyes closed (approximately 315 epochs), and EEG data can be collected for about 10 minutes with the subject's eyes open (approximately 315 epochs).

Block 408 is followed by block 410, in which EEG data with minimal artifacts are selected for further analysis, in this embodiment, a system, such as 102, can utilize various devices, techniques, and methodologies to screen collected EEG data for any artifacts, and if needed, modify or remove any affected epochs.

Block 410 is followed by block 412, in which the EEG data is analyzed using at least one averaging-type methodology. In the embodiment shown, a fractal dimension method is applied by a system, such as 102, to the collected EEG data. The fractal dimension method measures the complexity of a geometrical object, usually fractal (self-similar) in nature. The object can be defined by the formula N=r^{D} or equivalently D=log(N)/log(r). If an object has a fractal dimension D, and its linear scale is reduced by a factor of r in every spatial dimension, the measure of its length, area, or volume will increase by a factor of N (when measured in terms of the new scale). For the instance of a purely linear, Euclidean object, such as a line, square, or cube, this dimension will take an integer value (1, 2, or 3); the value found for the length of a straight line is independent of the scale of the measuring device. In the instance of a non-linear object, such as a fractal curve, the British coastline for example, or an EEG time series, this D will not have an integer value. For example, if measurements of the British coastline are taken with a ruler of a given length, and then subsequent measurements can be taken with a ruler of half that length, the second measure can provide an estimation of the coast length that is greater than the first. Thus a determination of D as D=log(N)/log(r) can be made. For the British coastline example, as well as any EEG time series, this can produce a D between 1 and 2. As another example, the Koch curve, also known as the snowflake fractal, has a fractal dimension of about 1.26. If you reduce the linear scale by a factor of 3, its length increases by a factor of 4, thus 4=3^{D} and D=log(4)/log(3)=1.26.

One suitable algorithm for use with the embodiment shown to measure the fractal dimension of an EEG time series is a box counting (BC) algorithm. The BC algorithm can cover a time series with a shrinking grid of boxes and count the number of boxes in the grid that contain at least one point of the series. This algorithm can be performed for each site at which the EEG data is recorded, a predefined number of sites, or at selected sites for each epoch analyzed after artifacting. Each epoch in the embodiment shown includes about 256 data points.

Using the raw EEG data, without first normalizing the data, the distance between two data points, and therefore the grid dimension, may have some or no meaning due to the difference in scale and units on the axes. Thus, prior to initializing the BC algorithm, the EEG data from that particular site and epoch should be normalized. The time data can be converted to units of about two seconds, thus time runs from zero to one instead of zero to two, which is the length in seconds of an epoch of EEG data for this algorithm. Each voltage value can be normalized by first subtracting the minimum data point from the given value, and then dividing the result by the range of the data, or as a formula:

$${v}_{norm} = \frac{v - {v}_{min}}{{v}_{max} - {v}_{min}}$$

This step can result in some or all data points in the set being in the unit square (time ranging from 0 to 1 on the x-axis, and normalized voltage ranging from 0 to 1 on the y-axis).

Once the data is normalized, a grid can be overlaid on some or all of the data set for the epoch under analysis. In the embodiment described above, electrode site, T5, is the site with optimal predictive power. For a 256-point epoch, the optimal range for a grid scale is approximately ¼ to 1/32, providing—from 16 to 1024 boxes, reduced by a factor of two each time (¼, ⅛, 1/16, 1/32). This range generally has a good linear correlation on the final log-log plot. The bottom and left sides of each box are not included in the box area, the top and right edges are, so that every data point is included in at least one box, but no point is included in more than one. Setting the grid in this manner can provide a relatively easy division of the time coordinates as each side length cleanly and evenly divides the time points (since 256=2⁸). When the number of boxes containing a point is calculated for each side length, the results can be plotted (ln(number) versus ln(1/side length)) and the slope of the resulting regression line of this plot can be the estimate of the fractal dimension for that epoch. The rationale for taking the reciprocal of the side length is that this changes the sign of the slope, thus providing the fractal dimension instead of its negative.

This process can be repeated for some or all epochs included after the artifacting process. The final estimate of the fractal dimension for a subject is the average of the fractal dimensions of included epochs. The averaging process described above can reduce the effects that any outlying data points may have on the overall fractal dimension for a subject, thus reducing the chance of significant error.

Block 412 is followed by block 414, which is described in greater detail below.

Neuropsychological or Cognitive Data Collection and Analysis. As shown in FIG. 4, the method 400 includes a neuropsychological or cognitive data collection and analysis sub-process 404. The sub-process 404 begins at block 416.

In block 416, neuropsychological or cognitive data is received from a subject. In the embodiment of FIG. 4, neuropsychological data can be received by conducting or administering a neuropsychological or cognitive test on a subject by, for example, a qualified professional. A suitable neuropsychological or cognitive test can include, but is not limited to, ADAS-Cog test. Neuropsychological or cognitive data can include, but is not limited to, data associated with memory, data associated with praxis, data associated with language skills, and ADAS-Cog-type data. In one embodiment, an ADAS-Cog test can be conducted on a subject by a medical professional or health care provider.

Block 416 is followed by block 418, in which a test score for the subject is calculated. In the embodiment of FIG. 4, a system, such as 102, can calculate or obtain a test score to represent some or all of the results of the neuropsychological test on the subject. For example, a subject's total neuropsychological or cognitive test score can provide suitable subject information for the method 400. For example, neuropsychological or cognitive test scores can be derived from data based in part on at least memory, praxis, and language skills of the subject. In one embodiment, an ADAS-Cog test score can be obtained by the system. In any instance, such test scores can be implemented with a logistic regression model as explained below. In another embodiment, individual ADAS-Cog memory variables can be implemented with a logistic regression model. In other embodiments, results from other types of neuropsychological or cognitive tests, such as memory tests, can be implemented with a logistic regression model. In one embodiment, a total ADAS-Cog test score can be implemented with a logistic regression model, and standardized against a database of scores. In one example, a database of scores can include scores associated with normal adults aged 50-85 years.

Block 418 is followed by block 420, in which the test score can be standardized using a normative database. In the embodiment shown, the system such as 102 can standardize the test score to a Z-score using a normative database. Those skilled in the art will recognize the techniques needed to standardize a test score against various types of databases or other collections of data.

Block 420 is followed by block 414, which is described in greater detail below.

Medical History Data Collection and Analysis. As shown in FIG. 4, the method 400 includes a medical history or risk factor data collection and analysis sub-process 406. The sub-process 406 begins at block 422.

In block 422, medical history associated with the subject can be received. In the embodiment shown, a system such as 102 can receive medical history associated with the subject. For example, medical history from a patient's files and a questionnaire can be collected and input to a system such as 102.

Block 422 is followed by block 424, in which at least one risk factor can be determined based at least in part on the collected medical data. In the embodiment shown, a system such as 102 can determine at least one cardiovascular risk factor by reference to some or all of the medical history associated with the subject, such as from a patient file and/or data collected in a questionnaire. In other embodiments, a system such as 102 can determine more than one cardiovascular risk factor or other similar type factors.

Risk factors can include, but are not limited to a cardiovascular risk factor, stroke, transient ischemic attack, myocardial infarct, alcohol abuse, arterial bypass surgery, and/or significant arterial blockage. Each of these risk factors has been previously demonstrated to be indicative of the relative risk of a person eventually suffering from AD and/or VAD (de la Torre, 2001). In one embodiment, some or all of these risk factors can be characterized as a cardiovascular risk factor.

In other embodiments, based at least in part on the collected medical data, a system such as 102 can determine at least one risk factor such as a series of cardiovascular risk factors and/or brain risk factors. Such risk factors can include, but are not limited to, hypertension, diabetes, untreated diabetes, age, smoking, head injury, migraine, gender, education level, body mass index, overweight, sedentary lifestyle, C-reactive protein, fibrinogen, lipoprotein (a), homocysteine, blood lipids, genetics, family history, high cholesterol, chronic obstructive pulmonary disease, emphysema, alcohol abstention, and not married (widowed, divorced, or single).

In another embodiment, a system such as 102 can determine at least one risk factor based at least in part on the collected medical data, such as brain imaging (MRI/CT) data, which is capable of detecting evidence of cardiovascular disease in a particular subject.

In another embodiment, a system such as 102 can determine at least one risk factor based at least in part on genetic testing data, such as the APOE-4 allele, which can be utilized to determine the probability of the subject developing dementia.

In yet another embodiment, a system such as 102 can determine at least one risk factor based at least in part on family history of dementia or similar disorders, which can provide suitable heredity information for a particular subject.

Block 424 is followed by block 414, which is described in greater detail below.

Integration Process and Analysis. In block 414, some or all of the data collection and analysis sub-processes 402, 404, 406 have been performed, some or all of the received or otherwise collected data is input into at least one statistical model. In the embodiment described in FIG. 4, data collection and analysis includes EEG data collection and analysis, neuropsychological or cognitive data collection and analysis, and medical history or risk factor data collection and analysis. In one example, a system such as 102 can input various data including, for example, one EEG data recording of about 10 minutes of eyes closed resting data measured from a T5 site on a subject's body with variables such as complexity calculated from the EEG data, data from a review and/or questionnaire covering the subject's relevant medical history with each risk factor entered as a dichotomous variable with a value of 0 if the specific risk factor is absent and a value of 1 if the specific risk factor is present, and data from the subject's neuropsychological or cognitive test (ADAS Cog) with scores calculated from the test such as total score into a logistic regression model or another suitable statistical-type model.

Block 414 is followed by block 426, in which a probability for dementia in the subject is determined. In the embodiment described in FIG. 4, a system such as 102 can determine an output or other indication from a logistic regression model such as a probability measure that a particular subject of interest is suffering from dementia (AD and/or VAD), mild cognitive impairment (MCI), or other dementia-type disorder. In one embodiment, a probability result can be interpreted by for example, a clinician using an ROC curve representing a clinical database, such as a database with data associated with dementia patients and normal adults aged 50-85 years. In that embodiment, the ROC curve and associated table can provide sensitivity and specificity results, which can be interpreted by a clinician when integrating the result together with the clinician's complete clinical evaluation and laboratory tests. In one embodiment, a clinician can select a single probability cutoff as a screen for dementia patients. For example, the clinician can select a cutoff of probability of approximately 0.5 to screen for dementia patients versus normal adults. Using calculations with a clinical database, such as a database with data associated with dementia patients and normal adults aged 50-85 years, the approximately 0.5 cutoff can provide a positive predictive power of about 85% and a negative predictive power of about 94%. In one embodiment, a clinician can select at least two probability cutoffs: one cutoff to represent the majority of the normal adult distribution and one cutoff to represent the majority of the dementia patient distribution. For example, using calculations with a clinical database such as a database with data associated with dementia patients and normal adults aged 50-85 years, selecting a cutoff of probability less than about 0.2 as a screen for normal adults can provide a negative predictive power of about 97%. Furthermore, the selection of a cutoff of probability greater than about 0.8 for a dementia screen can provide a positive predictive power of about 100%. All remaining subjects with probability values of greater than about 0.2 and less than about 0.8 can be designated by the clinician as “inconclusive”, “at risk”, or a similar term.

In block 428, a probability value can be returned or otherwise output, and the method 400 ends.

While the above description contains many specifics, these specifics should not be construed as limitations on the scope of the invention, but merely as exemplifications of the disclosed embodiments. Those skilled in the art will envision many other possible variations that are within the scope of the invention.

The claimed invention is:

1. A method for analyzing a dementia-type disorder in a person, comprising:
receiving a plurality of electroencephalography data associated with a person;
receiving a plurality of cardiovascular risk factor data associated with the person;
receiving a plurality of cognitive data associated with the person;
based at least in part on a portion of the electroencephalography data, cardiovascular risk factor data, and cognitive data, determining an indication of whether the person is at risk for the dementia-type disorder.

2. The method of claim 1, wherein the plurality of electroencephalography data can comprise at least one of the following: electroencephalography data taken at a T5 electrode site for the person, electroencephalography data collected with the person's eyes open, electroencephalography data collected with the person's eyes closed, or a combination of electroencephalography data collected with the person's eyes open and closed.

3. The method of claim 1, wherein at least a portion of the electroencephalography data is processed using at least one of the following: a fractal dimension methodology, or a box counting algorithm.

4. The method of claim 1, wherein the plurality of cardiovascular risk factor data can comprise any factor indicative of a higher probability for the person eventually suffering from cardiovascular disease associated with a history of at least one of the following: stroke, transient ischemic attack, myocardial infarct, alcohol abuse, arterial bypass surgery, arterial blockage, hypertension, high cholesterol, diabetes, untreated diabetes, chronic obstructive pulmonary disease, emphysema, alcohol abstention, overweight, male gender, and not married (widowed, divorced, or single).

5. The method of claim 1, wherein the plurality of cognitive data can comprise at least one of the following: an ADAS-Cog test score associated with the person, data associated with an ADAS-Cog test administered to the person, data associated with memory of the person, data associated with praxis of the person, or data associated with a language skill of the person.

6. The method of claim 1, wherein the dementia-type disorder can comprise at least one of the following: Alzheimer's disease (AD), vascular dementia (VAD), mixed dementia (AD and VAD), or mild cognitive impairment (MCI).

7. The method of claim 1, further comprising:
receiving a plurality of other health data associated with the person; and
based at least in part on a portion of the electroencephalography data, cardiovascular risk factor data, cognitive data, and other health data, determining an indication of whether the person is at risk for the dementia-type disorder.

8. The method of claim 7, wherein the other health data comprises at least one of the following: medical history of the person, health data collected from a questionnaire, brain imaging data, or genetic testing data.

9. A system for analyzing a dementia-type disorder in a person, comprising:
a data collection module adapted to:
receive a plurality of electroencephalography data associated with a person:
receive a plurality of cardiovascular risk factor data associated with the person;
receive a plurality of cognitive data associated with the person; and
a report generation module adapted to:
determine an indication of whether the person is at risk for the dementia-type disorder based at least in part on a portion of the electroencephalography data, cardiovascular risk factor data, and cognitive data.

10. The system of claim 9, wherein the data collection module is further adapted to receive a plurality of other health data associated with the person; and the report generation module is further adapted to determine an indication of whether the person is at risk for the dementia-type disorder based at least in part on a portion of the electroencephalography data, cardiovascular risk factor data, cognitive data, and other health data.

11. The system of claim 9, wherein the data collection module is further adapted to output the indication comprising a probability against a receiver operating characteristic (ROC) curve comprising data associated with a clinical database.

12. The system of claim 9, wherein the data collection module is further adapted to normalize some or all of the electroencephalography data.

13. The system of claim 9, wherein the data collection module is further adapted to implement an averaging methodology to some or all of the electroencephalography data.

14. The system of claim 9, wherein the data collection module is further adapted to implement a fractal dimension methodology to some or all of the electroencephalography data.

15. The system of claim 9, wherein the data collection module is further adapted to implement a box counting algorithm to some or all of the electroencephalography data.

16. The system of claim 9, wherein the data collection module is further adapted to implement a logistic regression model with some or all of the electroencephalography data.

17. The system of claim 9, wherein the data collection module is further adapted to implement a logistic regression model with some or all of the cognitive data.

18. The system of claim 9, wherein the data collection module is further adapted to standardize some or all of the cognitive data using a normative database.

19. The system of claim 9, wherein the data collection module is further adapted to implement a logistic regression model with some or all of the cardiovascular risk factor data.

20. A system for analyzing a dementia-type disorder in a person, comprising:
at least one data collector adapted to:
receive a plurality of electroencephalography data associated with a person;
receive a plurality of cardiovascular risk factor data associated with the person,
receive a plurality of cognitive data associated with the person;
at least one processor adapted to:
determine an indication of whether the person is at risk for the dementia-type disorder based at least in part on a portion of the electroencephalography data, cardiovascular risk factor data, and cognitive data; and
at least one output device adapted to:
output the indication of whether the person is at risk for the dementia-type disorder.

21. The system of claim 20, wherein the plurality of electroencephalography data can comprise at least one of the following: electroencephalography data taken at a T5 electrode site for the person, electroencephalography data collected with the person's eyes open, electroencephalography data collected with the person's eyes closed, or a combination of electroencephalography data collected with the person's eyes open and closed;
wherein the plurality of cardiovascular risk factor data can comprise any factor indicative of a higher probability for the person eventually suffering from cardiovascular disease associated with a history of at least one of the following, stroke, transient ischemic attack, myocardial infarct, alcohol abuse, arterial bypass surgery, arterial blockage, hypertension, high cholesterol, diabetes, untreated diabetes, chronic obstructive pulmonary disease, emphysema, alcohol abstention, overweight, male gender, and not married (widowed, divorced, or single); and
wherein the plurality of cognitive data can comprise at least one of the following: an ADAS-Cog test score associated with the person, data associated with an ADAS-Cog test administered to the person, data associated with memory of the person, data associated with praxis of the person, or data associated with a language skill of the person.
